(19) United States
(12) Patent Application Publication
(10) Pub. No.: US 2002/0127251 A1
(43) Pub. Date: Sep. 12, 2002

(54) Cosmetic or pharmaceutical compositions comprising thermally stabilizing microcapsules

(76) Inventors: Bruno Biatry, Vincennes (FR); Jean Mondet, Aulnay-Sous-Bois (FR); Isabelle Bara, Paris (FR); Isabelle Afriat, Paris (FR)

Correspondence Address: STEPTOE & JOHNSON LLP, 1330 Connecticut Avenue, N.W., Washington, DC, 20036, US

(21) Appl. No.: 09/983,762

(22) Filed: Oct. 25, 2001

(30) Foreign Application Priority Data
Oct. 27, 2000 (FR) 0013792
Oct. 27, 2000 (FR) 0013793

(51) Int. Cl. A61K 7/00; A61K 9/16; A61K 9/50

(52) U.S. Cl. 424/401; 424/490

(57) ABSTRACT

The present invention relates to the use of thermally stabilizing microcapsules including at least one crystalline compound exhibiting an enthalpy of fusion (ΔH_{f}), measured by differential scanning calorimetry, of between 75 and 330 kJ/kg for protecting cosmetic or pharmaceutical compositions against the effects of thermal variations and to the cosmetic or pharmaceutical compositions comprising such thermally stabilizing microcapsules.

The present invention relates to the use of thermally stabilizing microcapsules including thermally absorbing crystalline compounds for protecting cosmetic or pharmaceutical compositions against the effects of thermal variations and to the cosmetic or pharmaceutical compositions comprising such thermally stabilizing microcapsules.

The problem is frequently encountered, in the field of the formulation of cosmetic and pharmaceutical compositions, of the rheological instability of cosmetic compositions when the latter are exposed to significant variations in temperature.

This is because numerous compositions are thickened or gelled in a thermally reversible fashion, that is to say an increase in the external temperature tends to liquefy or soften them and a cooling can be reflected by an undesirable hardening or thickening of the composition.

Thus, the applicational properties of lipsticks, of colouring pencils or of deodorant sticks can be found to be detrimentally affected because their consistency has become too soft or too hard following a change in temperature. A sun cream having a satisfactory viscosity at ambient temperature can excessively liquefy under the effect of heat, which renders its application more difficult.

Furthermore, rheological variations can in some cases lead to irreversible defects, such as heat deformation of a solid composition or phase separation in the case of compositions in the dispersed form. Emulsions and suspensions can also be destabilized during a fall in temperature.

A specific case of cosmetic or pharmaceutical compositions to be stabilized against thermal variations are compositions for the impregnation of wipes, such as, for example, make-up-removing wipes. These compositions are generally oil-in-water emulsions. In order for the process of impregnation of the wipes by spraying or by steeping to be able to be carried out under satisfactory conditions, the make-up-removing milk has to be sufficiently fluid. The need for a low viscosity prevents the introduction of significant amounts of emulsion-stabilizing polymers, such as Carbomer® products. The impregnation compositions and impregnated wipes are thus particularly sensitive to thermal variations and in particular the phenomenon of creaming of emulsions (separating by settling of oily globules, leading to the formation of an aqueous phase devoid of oily globules) is promoted at high temperature.

It is thus important to find a solution in order to retain good stability of the emulsion at high temperatures while remaining within low viscosity ranges which allow the impregnation of wipes according to conventional techniques.

Furthermore, in the cosmetics field, numerous situations are encountered where it is advantageous to control thermal exchanges between the body, the cosmetic composition and the environment.

Thus, the exposure of human skin to very high or very low temperatures can lead to undesirable effects, such as drying out or red blotches.

The protection of the skin and lips against cold is generally carried out by contributing fatty substances, which is generally not very satisfactory from a cosmetic viewpoint.

The protection of the skin against excess heat also proves to be difficult. A known approach consists in adding menthol or derivatives of the latter to the compositions, which products, by a biological effect, bring about a local and transitory feeling of coolness. Direct contact between the chemical (menthol) and the skin can, however, lead to cutaneous irritation.

The quality and the longevity of the make-up often depend on specific thermal conditions and can be highly detrimentally affected under extreme temperature conditions. Deposits of products can either liquefy and run under the effect of heat and of sweat or, in the case of extreme cold, can lose their plasticity and their flexibility.

No effective solution to the stated problems has been introduced to date.

The Applicant Company has discovered, surprisingly, that it is possible to substantially improve the thermal stability of cosmetic and pharmaceutical compositions and to at least partially solve the problems described above, related to thermal exchanges in the cosmetic field, by virtue of the use of specific microcapsules, described in more detail below, having heat absorption and restoration capabilities.

The microcapsules used in the present invention preferably comprise, in a leaktight covering, partially or completely crystalline compounds which, when they are brought to a temperature close to their melting point, absorb a significant amount of heat, known as the latent heat of fusion. The absorption of this latent heat of fusion is reflected by the temperature of the compound remaining stable despite the introduction of thermal energy. This effect is similar to a <<buffer>> effect and makes it possible to thermostatically control, for a certain period of time and in a temperature range close to the melting point of the compound, the immediate environment of the microcapsules, despite a variation in the external temperature.

The heat absorption capability described above goes together with the possibility, also highly advantageous, of restoration the absorbed energy in the form of latent heat of crystallization when the temperature is lowered below the melting point of the encapsulated compound. The microcapsules comprising the compound in the molten state thus constitute a reserve of thermal energy.

The microcapsules which have reversible heat absorption and restoration capabilities and which are capable of stabilizing their immediate environment will subsequently be known as <<thermally stabilizing microcapsules>>.

A subject-matter of the present invention is consequently the use of microcapsules including at least one crystalline compound exhibiting an enthalpy of fusion (ΔH_{f}), measured by differential scanning calorimetry, of between 75 and 330 kJ/kg for protecting cosmetic or pharmaceutical compositions against the effects of thermal variations.

Another subject-matter of the invention is cosmetic or pharmaceutical compositions comprising, in a physiologically acceptable vehicle, microcapsules including at least one crystalline compound exhibiting an enthalpy of fusion (ΔH_{f}), measured by differential scanning calorimetry, of between 75 and 330 kJ/kg.

The introduction of thermally stabilizing microcapsules into cosmetic or pharmaceutical compositions makes it possible to protect the latter for a certain period of time against episodes of sudden heat or cooling, that is to say to retain their rheological characteristics and to prevent the irreversible effects related to changes in temperature.

The effectiveness of the thermally stabilizing microcapsules used in the present invention for improving the thermal stability of cosmetic or pharmaceutical compositions depends directly on the thermal absorption capability of the encapsulated crystalline compounds and on their melting point.

The term <<crystalline compound>> as used in the present application encompasses partially or completely crystalline compounds. The degree of crystallinity of the compounds used is not determining in so far as the compound exhibits the enthalpy of crystalline fusion required for the envisaged use.

The thermal absorption capability of the microcapsules used is directly proportional to the enthalpy of fusion of the encapsulated crystalline compound. This enthalpy of fusion is measured by differential scanning calorimetry.

The enthalpy of fusion of a compound is the amount of energy necessary to convert a partially or completely crystalline sample into a completely amorphous sample. The thermogram ΔCp=f(T), in which ΔCp represents the difference in thermal capacity of the sample with respect to a reference sample which does not undergo any thermal transition in the range studied, thus exhibits an endothermic signal with an area proportional to the enthalpy of fusion (ΔH_{f}) of the sample.

As indicated, microcapsules used in the cosmetic compositions of the present invention comprise crystalline compounds having an enthalpy of fusion of between 75 and 330 kJ/kg. This enthalpy of fusion is preferably between 100 and 300 kJ/kg and ideally between 150 and 280 kJ/kg.

In addition to the thermal capacity of the crystalline compounds included in the thermally stabilizing microcapsules of the cosmetic or pharmaceutical compositions, the melting point of these compounds is an essential parameter determining the method of action, the field of application and the effectiveness of the cosmetic compositions.

For the protection of cosmetic or pharmaceutical compositions against thermal variations, the melting of the encapsulated crystalline compound must take place at temperatures to which the cosmetic compositions are liable to be exposed, namely, preferably, in a temperature range of between approximately 0° C. and 90° C.

When it is a matter of protecting solid compositions, such as sticks or pencils, against excessively great hardening under cold conditions, the encapsulated compound must have a melting point of less than ambient temperature, for example of between 5° C. and 15° C., whereas the protection of the compositions against softening or liquefaction under warm conditions requires encapsulated compounds having a melting point of greater than ambient temperature, for example of between 25 and 60° C.

As regards the thermal exchanges mentioned in the introduction between the skin and cosmetic and pharmaceutical compositions, other aspects have to be taken into consideration for the choice of the melting point.

Thus, in one embodiment of the cosmetic and pharmaceutical compositions of the present invention, the melting point (Tm) of the encapsulated crystalline compound is less than ambient temperature, preferably of between 0 and 15° C.

Microcapsules including crystalline compounds with a relatively low melting point in a protection cream make it possible to protect the skin against attack by cold by virtue of the release of the latent heat of crystallization of the encapsulated compound when the external temperature falls below the melting point of the encapsulated crystalline compound.

In another embodiment of the cosmetic and pharmaceutical compositions of the present invention, the melting point of the encapsulated crystalline compound is between ambient temperature, that is to say approximately 20 to 25° C., and the temperature of the human skin, that is to say approximately 32° C. At the storage temperature of these compositions, the crystalline compound encapsulated in the microcapsules is in the crystalline state. It will rapidly melt after application to the skin or lips, drawing the heat necessary for this melting from the immediate cutaneous environment and thus creating, in the user, a local feeling of coolness.

This coolness effect, comparable to that of menthol, does not, however, involve direct contact between the chemical and the skin and thus excludes any risk of irritation.

In a third embodiment of the cosmetic and pharmaceutical compositions of the present invention, the melting point of the encapsulated crystalline compound is greater than the temperature of the human skin.

Such compositions will make it possible, in the temperature range close to the melting point of the encapsulated compound, to prevent the harmful effects of heat.

The incorporation of thermally stabilizing microcapsules including a compound having a melting point slightly greater than the temperature of the human skin, for example of between 33 and 45° C., is particularly advantageous in the field of make-up. During an increase in the external temperature, the encapsulated crystalline compound will melt, locally stabilizing for a certain period of time the temperature at a value close to its melting point.

This stabilization of the temperature improves the hold of the make-up and preserves its cosmetic qualities, for example the mattness of the colouring, preventing it from running and locally limiting perspiration, indeed even the rate of secretion of the sebaceous glands.

It is, of course, possible to combine, in the same cosmetic composition, several types of microcapsules which differ in the melting point of the compound which they include.

The above listing of the applications of the cosmetic compositions comprising the thermally stabilizing microcapsules of the present invention is, of course, not limiting. The <<thermostatically controlled>> cosmetic compositions of the present invention will be of use in any application where the local introduction or removal of heat or the local stabilization of the temperature of the surfaces (skin, lips or hair) to which they are applied is concerned.

The crystalline compounds are preferably encapsulated in a leaktight covering.

This encapsulation is an essential condition for the reversibility of the melting/crystallization methods. This is because the encapsulated molten compound cannot diffuse into the cosmetic or pharmaceutical composition and will crystallize again when the external temperature becomes less than its melting point.

The leaktightness of the covering is advantageous in particular when the composition is liable to be exposed to numerous thermal variations in the melting range of the crystalline compound.

It is essential, for example, in creams for protecting against cold comprising, at ambient temperature, the crystalline compound in the molten state.

However, in some applications, in particular when a single thermal stabilization by melting of the crystalline compound is desired, the reversibility of the melting/crystallization phenomena is not essential and the impermeability of the covering with respect to the molten crystalline compound is not an essential characteristic of the invention.

Mention may be made, as examples of encapsulated crystalline compounds appropriate for the present invention, of

aliphatic hydrocarbons comprising from 10 to 40 carbon atoms,

aromatic hydrocarbons,

C₈₋₄₀ fatty acids, such as stearic acid and lauric acid,

C₈₋₄₀ fatty alcohols, such as stearyl alcohol and lauryl alcohol,

C₁₀₋₄₀ fatty acid esters, such as methyl stearate and methyl cinnamate,

inorganic salts comprising a significant fraction of water of crystallization, such as calcium chloride hexahydrate, sodium sulphate decahydrate, sodium hydrogenphosphate dodecahydrate, sodium thiosulphate pentahydrate and nickel nitrate hexahydrate,

C₁₀₋₄₀ fatty acid triglycerides,

some silicone waxes, such as polydimethylsiloxanes comprising behenoxy or stearoxy end groups (INCI: behenoxydimethicone and stearoxydimethicone), poly-methylstearyloxydimethylsiloxanes (INCI: stearic ester dimethicone), polymethylstearyldimethylsiloxanes (INCI: stearyldimethicone), copolymers comprising stearyl methacrylate units with polydimethylsiloxane grafts, or polymethyltrifluoromethylalkyldimethylsiloxanes (INCI: trifluoromethyl(C₁₋₄) alkyldimethicone),

the fatty chains of all these compounds preferably being linear and/or saturated.

These compounds can be used alone or in the form of a mixture of two or more of them.

Mention may also be made of at least partially crystalline hot-melt polymers exhibiting a crystalline melting point in the temperature region indicated above.

Such polymers are, for example, olefinic homopolymers and copolymers, including polyolefin waxes, such as ethylene homopolymers, copolymers of ethylene and of propylene, copolymers of ethylene and of octene, copolymers of ethylene and of butene, and copolymers of ethylene and of vinyl acetate.

Mention may also be made of poly(alkylene oxide)s, poly(alkyl ester)s, poly(ε-caprolactone)s, polyamides, in particular those resulting from the polycondensation of a dimer of fatty acids, and copolymers of fluoroolefins.

Another group of crystalline polymers which can be used is formed of the polymers with crystallizable side chains described in J. Polymer Sci.: Macromol. Rev., 8, 117-253 (1974). They are vinyl and/or acrylic polymers or copolymers comprising a significant fraction, generally at least equal to 50% by weight, of copolymerized units comprising long crystallizable linear aliphatic side chains or comprising crystallizable fluorinated or perfluorinated side chains. U.S. Pat. No. 5,156,911 discloses the use of such polymers with crystallizable side chains in adhesive assemblies, the adhesive properties of which vary as a function of the temperature.

The encapsulated crystalline compounds preferred for the present invention are aliphatic hydrocarbons with a linear chain comprising from 10 to 40, preferably from 13 to 28 and better still from 16 to 23 carbon atoms. This is because the melting point of the compounds of this series of homologous hydrocarbons increases in a completely predictable way with the number of carbon atoms (−5.5° C. for n-tridecane(C₁₃) to 61.4° C. for n-octacosane (C₂₈)) in the temperature range relating to the cosmetic applications described above.

The crystalline compounds exhibiting an enthalpy of fusion (ΔH_{f}) of between 75 and 330 kJ/kg are preferably encapsulated in a leaktight covering.

This encapsulation in an essential condition for the reversibility of the melting/crystallization processes. This is because the encapsulated molten compound cannot diffuse into the cosmetic composition and will crystallize again when the external temperature becomes less than its melting point.

The material forming the wall of the microcapsules can be chosen from any material conventionally used in the field of microencapsulation.

This material can be amorphous, crystalline or semicrystalline. When it is crystalline or semicrystalline, it must have a melting point greater than that of the encapsulated crystalline compounds.

Furthermore, this material must be sufficiently elastic to withstand the variations in volume of the crystalline compound during the phase transition and to withstand the shear forces during the application of the composition comprising it. Moreover, it must be inert with respect to encapsulated substances and with respect to compounds of the cosmetic or pharmaceutical formulation with which it will be in contact.

Depending upon the method chosen, use may be made, as materials, of polymers, such as polyamides, polyurethanes, polyureas, polyesters or polycyanoacrylates, urea-formaldehyde or melamine-formaldehyde resins, and gelatin/gum arabic systems.

The microcapsules can be prepared according to well-known methods, for example described in the work entitled <<Microencapsulation, Methods and Industrial Applications>>, Publishing Manager S. Benita, Marcel Dekker (1996). Mention may be made, by way of examples, of interfacial polymerization or polycondensation, coacervation, atomization, centrifugal extrusion or rotary disc microencapsulation.

Thermally stabilizing microcapsules are known and are sold, for example under the name Thermasorb® by Frisby Technologies Inc. or under the references 9850K and 9850Q by 3M.

These microcapsules are provided in the form of a fine, fluid and non-film-forming powder. Their use is known, for example, in the field of isothermal clothing and footwear, in cooling systems in microelectronics, and in the field of packaging.

A nonpolymeric material can also be used as compound constituting the wall of the microparticles. Use may be made, for example, of microcapsules based on precipitated silica, amorphous silica, hydrated silica or silica which has been rendered hydrophobic which are provided by Phase Change Laboratories under the name AcuTemp®.

The upper size of the microcapsules is preferably limited, for obvious reasons of visibility, to a few tens or hundreds of micrometers. It is generally preferable to use microcapsules having a mean diameter of between 0.01 and 100 micrometers, better still between 0.05 and 50 micrometers.

The proportion of the microcapsules in the cosmetic or pharmaceutical compositions of the present invention can vary between wide limits which depend on the formulation and on the application envisaged.

The cosmetic compositions of the present invention generally comprise from 1% to 99% by weight and preferably from 5% to 90% by weight of thermally stabilizing microcapsules, with respect to the final cosmetic or pharmaceutical composition.

The cosmetic or pharmaceutical compositions can additionally comprise adjuvants and cosmetic and/or pharmaceutical active principles appropriate for the application envisaged. They preferably comprise at least one material with optical properties. This material can be chosen, for example, from water-soluble or fat-soluble dyes, white or coloured pigments, lakes, polymer powders, pearlescent agents or glitter.

These materials are preferably present in the compositions of the present invention in an amount of between 0.1 and 99% by weight, preferably between 0.5 and 90% by weight.

Mention may be made, as examples of cosmetic active principles, of agents for combating free radicals, moisturizing agents, vitamins, proteins, enzymes, ceramides, a-hydroxy acids, P-hydroxy acids, retinoids, sunscreens, surfactants, mattness agents, antiperspirants, bactericidal and bacteriostatic agents, fatty substances and silicones.

The adjuvants are, for example, solvents, pH-regulating agents, antioxidants, sequestering agents, preservatives, fillers, emollients, antifoaming agents, fatty substances, such as oils, waxes and pasty fatty substances, dispersing agents, silicones, such as volatile or nonvolatile oils, gums, waxes or pasty silicones, fragrances, surfactants, plasticizers, thickening or gelling polymers, and film-forming polymers which are soluble or dispersible.

The cosmetic or pharmaceutical compositions of the present invention can be provided in any known form compatible with the presence of the thermally stabilizing microcapsules. They can, for example, be in the form of an aqueous, aqueous/alcoholic or organic suspension, in the form of an oil-in-water or water-in-oil emulsion or in the form of a multiple emulsion, or in the form of an aqueous or oily gel, of a paste, of a free or compact powder, or of a stick.

Another subject-matter of the present invention is insoluble solid substrates impregnated with a cosmetic or pharmaceutical composition as described above.

This is because the cosmetic or pharmaceutical compositions of the present invention can also be used to impregnate an insoluble solid substrate. The insoluble substrate can be chosen from the group consisting of textiles, whether woven or nonwoven, foams, sponges, wadding, and beads. It can in particular be a nonwoven textile substrate based on fibres of natural origin, such as linen, cotton or silk fibres, or of synthetic origin, such as cellulose fibres, viscose fibres, vinyl polymer fibres, polyester fibres, such as poly(ethylene terephthalate) fibres, polyolefin fibres, such as polyethylene or polypropylene fibres, polyamide fibres, such as Nylon® fibres, or acrylic polymer fibres. Nonwoven materials are described, for example, in <<Nonwoven Binding Methods & Materials>>, by Riedel, Nonwoven World, 1987. These substrates are obtained according to methods known in the field of the art of preparing nonwovens.

This substrate can comprise one or more layers having identical or different properties and which can contribute, for example, properties of elasticity or of softness depending upon the use envisaged. The substrates can, for example, comprise two parts having different properties of elasticity, such as those disclosed in International Application WO 99/13861, or else can comprise a single layer with different densities, as disclosed in the document WO 99/25318, or alternatively can comprise two layers of different texture, such as the substrates disclosed, for example, International Application WO 98/18441.

The substrate can have any size or shape appropriate to the envisaged application.

It generally has a surface area of between 0.005 m² and 0.1 m², preferably between 0.01 m² and 0.05 m². It is preferably provided in the form of rectangular wipes or of round compresses.

The final article comprising the substrate and the impregnation composition is generally in the moist state, with a degree of impregnation, that is to say an amount of composition with respect to the weight of the solid substrate, of between 200 and 1 000%, preferably between 250 and 350%.

The techniques for impregnating substrates are well-known in the technical field and are all applicable to the present invention. Generally, the impregnation composition is added to the substrate by one or more techniques, such as immersion, coating or vaporization.

It is also possible to form an article (or wipe) presented in the dry state, either by removing the water from the composition after it has been impregnated on the substrate or by depositing, on the substrate, a composition in the dry form, in the state of a powder, granule or film, by any known preparation means, such as the welding and the adhesive bonding of multilayers by the thermal route or by ultrasound. In the latter embodiment, the composition is dried by any known means, for example by atomization, lyophilization or any other analogous technique.

It is thus possible to obtain, depending upon the use envisaged, wet wipes or dry wipes. Wet wipes can be used as such, whereas dry wipes are wetted before use.

As indicated above, the compositions comprising thermally stabilizing microcapsules of the present invention have numerous potential applications.

They can, for example, be make-up compositions, such as foundations, powders, mascaras, eyeliners, lipsticks or products for making up the hair, deodorant products, in particular in the form of sticks or cream, protection creams, care creams or haircare products.

The articles or wipes with an insoluble solid support described above can in particular be articles or wipes appropriate for caring for and/or treating the skin and in particular a wipe for cleansing or removing make-up from the skin of the face and/or the body and/or a wipe for cleaning or removing make-up from the eyes, and for cleansing skin with a tendency towards greasiness or acne.

As indicated above, the thermal buffer effect of the thermally stabilizing microcapsules used in the present invention is particularly advantageous in the case of compositions liable to be subjected to a detrimental reversible or irreversible change in their properties following a change in temperature. Mention may be made, by way of examples of such compositions, of deodorant sticks, solid scenting compositions, lipsticks, make-up pencils, suncreams, mascaras, care creams, aqueous or oily gels, and wipes impregnated with an oil-in-water emulsion, such as make-up-removing wipes.

The examples below illustrate the present invention without, however, limiting it.

### EXAMPLE 1

### Thermal Stabilization of a Cosmetic Composition

The change in the stability of anhydrous sticks as a function of the external temperature is observed. The sticks tested have the following composition:

| Stick A (control): | |
|---|---|
| Lanolin | 24% by weight |
| Lanolin derivative | 5.95% by weight |
| Lanolin wax | 6% by weight |
| Fatty acid ester | 4% by weight |
| Modified clay | 0.6% by weight |
| Pigments | 8.66% by weight |
| Antioxidizing agent | 0.6% by weight |
| Fragrance | 0.2% by weight |
| Vegetable oil q.s. for | 100% by weight |

Stick B (according to the invention)

65% by weight of the composition forming stick A

±35% by weight of icosane-comprising microcapsules.

The sticks are kept in a thermostatically-controlled oven at 47° C., they are removed from the oven at intervals of 10 minutes and the temperature of each of the sticks is measured immediately using an IR thermometer (Infratherm model from Chauvin/Arnoux, France).

The following results are obtained:

| | | T° of | T° of | T° of |
|---|---|---|---|---|
| | T° of | the | the | the |
| | the | stick | stick | stick |
| | stick | after | after | after |
| | at T₀ | 10 min | 20 min | 30 min |
| Stick A (control) | 27.6° C. | 35.0° C. | 37.7° C. | 38.5° C. |
| Stick B (invention) | 27.4° C. | 32.3° C. | 34.2° C. | 36.4° C. |

These results show that the presence of the microcapsules makes it possible to slow down the rise in temperature of the stick.

### EXAMPLE 2

A free powder is prepared by mixing the following ingredients:

| | |
|---|---|
| Microcapsules 9850Q¹⁾ | 40 parts by weight |
| Talc | 44 parts by weight |
| Nylon powder | 10 parts by weight |
| Yellow iron oxide | 1.6 parts by weight |
| Red iron oxide | 1.0 part by weight |
| Black iron oxide | 0.4 part by weight |
| Dimethicone²⁾ | 3 parts by weight |

¹⁾microcapsules, sold by 3M, including a compound with a melting point at 34° C.
²⁾nonvolatile polydimethylsiloxane oil

The free powder thus prepared makes it possible to maintain good mattness of the complexion at relatively high external temperatures. EXAMPLE 3

A free powder is prepared by mixing the following ingredients:

| | |
|---|---|
| Thermasorb ® 83 | 40 parts by weight |
| Microcapsule¹⁾ | |
| Talc | 44 parts by weight |
| Nylon powder | 10 parts by weight |
| Yellow iron oxide | 1.6 parts by weight |
| Red iron oxide | 1.0 part by weight |
| Black iron oxide | 0.4 part by weight |
| Dimethicone²⁾ | 3 parts by weight |

¹⁾microcapsules, sold by Frisby Technologies, including a compound with a melting point at 28° C.
²⁾nonvolatile polydimethylsiloxane oil

During the application of the powder, an immediate effect of coolness is found.

### EXAMPLE 4

### Make-Up-Removing Milk Composition for Wipes

| Phase A | |
|---|---|
| Glyceryl stearate/PEG 100 stearate | 0.55% |
| (Arlacel ® 165) | |
| Glyceryl stearate/PEG 100 stearate | 0.55% |
| (Arlacel ® 165) | |
| Cetyl alcohol | 0.15% |
| Xanthan gum | 0.1% |
| Isopropyl palmitate | 3.8% |
| Phase B | |
| Water q.s. for | 100% |
| Preservatives (parabens, phenoxyethanol) | 0.65% |
| Phase C | |
| Thermally stabilizing microcapsules | 10% |

Phases A and B are heated separately at a temperature of between 75 and 80° C. Phase A is added to phase B with stirring, stirring is maintained for 5 minutes and then phase C is added with gentle stirring. The mixture is allowed to cool to ambient temperature while maintaining gentle stirring. A milk with a low content of thickening polymer is thus obtained which exhibits good resistance to creaming at high temperature. This milk can be used for the impregnation of make-up-removing wipes according to conventional impregnation techniques.

1. Cosmetic or pharmaceutical composition comprising, in a physiologically acceptable vehicle, microcapsules including at least one crystalline compound exhibiting an enthalpy of fusion (AHt), measured by differential scanning calorimetry, of between 75 and 330 kJ/kg.

2. Cosmetic or pharmaceutical composition according to claim 1, characterized in that the microcapsules have a leaktight covering impermeable to the crystalline compound in the molten state.

3. Cosmetic or pharmaceutical composition according to claim 1 or 2, characterized in that the enthalpy of fusion of the crystalline compound is between 100 and 300 kJ/kg, preferably between 150 and 280 kJ/kg.

4. Cosmetic or pharmaceutical composition according to any one of the preceding claims, characterized in that the melting point (Tₘ) of the encapsulated crystalline compound is between 0° C. and 90° C.

5. Cosmetic or pharmaceutical composition according to claim 4, characterized in that the melting point of the encapsulated crystalline compound is between 5 and 15° C.

6. Cosmetic or pharmaceutical composition according to claim 4, characterized in that the melting point of the encapsulated crystalline compound is between 25 and 60° C.

7. Cosmetic or pharmaceutical composition according to claim 4, characterized in that the melting point (Tₘ) of the encapsulated crystalline compound is less than ambient temperature, preferably of between 0 and 15° C.

8. Cosmetic or pharmaceutical composition according to claim 4, characterized in that the melting point of the encapsulated crystalline compound is between ambient temperature and the temperature of the human skin (32° C.).

9. Cosmetic or pharmaceutical composition according to claim 4, characterized in that the melting point of the encapsulated crystalline compound is greater than the temperature of the human skin.

10. Cosmetic or pharmaceutical composition according to any one of the preceding claims, characterized in that the encapsulated crystalline compound is chosen from
aliphatic hydrocarbons comprising from 10 to 40 carbon atoms,
aromatic hydrocarbons,
C₈₋₄₀ fatty acids,
C₈₋₄₀ fatty alcohols,
C₁₀₋₄₀ fatty acid esters,
inorganic salts comprising a significant fraction of water of crystallization,
C₁₀₋₄₀ fatty acid triglycerides,
silicone waxes, the fatty chains of these compounds preferably being linear and/or saturated, and
crystalline hot-melt polymers.

11. Cosmetic or pharmaceutical composition according to any one of the preceding claims, characterized in that the crystalline compound is an aliphatic hydrocarbon with a linear and saturated chain comprising from 10 to 40 carbon atoms, preferably from 13 to 28 carbon atoms and better still from 16 to 23 carbon atoms.

1. Cosmetic or pharmaceutical composition according to any one of the preceding claims, characterized in that the material forming the covering is chosen from polyamides, polyurethanes, polyureas, polyesters, polycyanoacrylates, urea-formaldehyde or melamine-formaldehyde resins, gelatin/gum arabic systems and silica.

13. Cosmetic or pharmaceutical composition according to any one of the preceding claims, characterized in that the microcapsules have a mean diameter of between 0.01 and 100 micrometers, preferably between 0.05 and 50 micrometers.

14. Cosmetic or pharmaceutical composition according to any one of the preceding claims, characterized in that the microcapsules represent from 1 to 99% by weight, preferably from 5 to 90% by weight, of the final composition.

15. Cosmetic or pharmaceutical composition according to claim 1, characterized in that it additionally comprises at least one material with optical properties.

16. Cosmetic or pharmaceutical composition according to claim 15, characterized in that the material with optical properties is present in a proportion of 1 to 99% by weight, with respect to the final cosmetic composition.

17. Cosmetic or pharmaceutical composition according to any one of the preceding claims, characterized in that it is a make-up composition, a deodorant product, a protection cream, a care cream or a haircare product.

18. Use of microcapsules including at least one crystalline compound exhibiting an enthalpy of fusion (ΔH_{f}), measured by differential scanning calorimetry, of between 75 and 330 kJ/kg for protecting cosmetic or pharmaceutical compositions against the effects of thermal variations.

19. Use according to claim 18, characterized in that the microcapsules have a leaktight covering impermeable to the crystalline compound in the molten state.

20. Use according to claim 18 or 19, characterized in that the enthalpy of fusion of the crystalline compound is between 100 and 300 kJ/kg, preferably between 150 and 280 kJ/kg.

21. Use according to one of claims 18 to 20, characterized in that the melting point (Tₘ) of the encapsulated crystalline compound is between 0° C. and 90° C.

22. Use according to claim 21, characterized in that the melting point of the encapsulated crystalline compound is between 5 and 15° C.

23. Use according to claim 21, characterized in that the melting point of the encapsulated crystalline compound is between 25 and 60° C.

24. Use according to any one of claims 18 to 23, characterized in that the encapsulated crystalline compound is chosen from
aliphatic hydrocarbons comprising from 10 to 40 carbon atoms,
aromatic hydrocarbons,
C₈₋₄₀ fatty acids,
C₈₋₄₀ fatty alcohols,
C₁₀₋₄₀ fatty acid esters,
inorganic salts comprising a significant fraction of water of crystallization,
C₁₀₋₄₀ fatty acid triglycerides,
silicone waxes, the fatty chains of these compounds preferably being linear and/or saturated, and
crystalline hot-melt polymers.

25. Use according to one of claims 18 to 24, characterized in that the crystalline compound is an aliphatic hydrocarbon with a linear chain comprising from 10 to 40 carbon atoms, preferably from 13 to 28 carbon atoms and better still from 16 to 23 carbon atoms.

26. Use according to one of claims 18 to 25, characterized in that the material forming the covering is chosen from polyamides, polyurethanes, polyureas, polyesters, polycyanoacrylates, urea-formaldehyde or melamine-formaldehyde resins, gelatin/gum arabic systems and silica.

27. Use according to one of claims 18 to 26, characterized in that the microcapsules have a mean diameter of between 0.01 and 100 micrometers, preferably between 0.05 and 50 micrometers.

28. Insoluble solid substrate impregnated with a cosmetic or pharmaceutical composition according to any one of claims 1 to 17.

29. Insoluble solid substrate according to claim 28, characterized in that it is a nonwoven textile substrate based on fibres of natural or synthetic origin.

30. Insoluble solid substrate according to either of claims 28 and 29, characterized in that it has a surface area of between 0.005 m² and 0.1 m², preferably between 0.01 m² and 0.05 m².

31. Insoluble solid substrate according to one of claims 28 to 30, characterized in that the degree of impregnation, that is to say the amount of cosmetic or pharmaceutical composition with respect to the weight of the solid substrate, is between 200% and 1 000%, preferably between 250% and 350%.

32. Insoluble solid substrate according to one of claims 28 to 31, characterized in that it is provided in the form of rectangular wipes or of round compresses.
